(19) United States
(12) Patent Application Publication
(10) Pub. No.: US 2026/0061141 A1
(43) Pub. Date: Mar. 5, 2026

(54) EPIDERMIS ANCHORING SYSTEM FOR DELIVERY TOOL

(71) Applicant: ClearPoint Neuro, Inc., Solana Beach, CA (US)

(72) Inventors: Ernesto Salegio, Solana Beach, CA (US); Jeremy Stigall, Solana Beach, CA (US); Princeton Saroha, Solana Beach, CA (US)

(21) Appl. No.: 19/319,369

(22) Filed: Sep. 4, 2025

Related U.S. Application Data
(60) Provisional application No. 63/690,451, filed on Sep. 4, 2024.

(51) Int. Cl. A61M 5/32; A61M 25/02

(52) U.S. Cl. CPC A61M 5/3287; A61M 2025/028; A61M 2210/1003

(57) ABSTRACT

An anchor system is configured for securing a delivery tool to skin. The system includes an outer housing forming an internal lumen along an axis with a distal foot with a surface configured to be secured to a patient’s skin. A carriage sleeve is slidably positioned within the internal lumen. A distance gauge on the carriage sleeve is visible through a window for providing a user an indication of a distance of movement of the carriage sleeve. An actuator is coupled to the outer housing, wherein the actuator is actuated to slidably move the carriage sleeve through the internal lumen.

### CROSS REFERENCE TO RELATED APPLICATIONS

This application claims the benefit of priority under 35 U.S.C. §119(e) to U.S. Provisional Patent Application Serial No. 63/690,451, filed September 4, 2024. The disclosure of the application is incorporated by reference in its entirety.

### BACKGROUND

Delivery of biologics to targeted areas of the nervous system requires the localization of a biologics delivery tool, such as a distal tip of the delivery tool, near the biological area of interest or in route to the area of interest. A flexible catheter or rigid catheter or cannula are examples form factors of such delivery tools. The delivery tool can span various lengths configured to extend to a target area of interest in tissue including just past the target tissue or extending further past the target tissue and down the vascular system. Dosing of the biologics takes place through a distal exit port of a lumen enclosed within the delivery system of choice.

### SUMMARY

Disclosed is a delivery system, or a delivery system component, that enables safe and secured external anchoring of a device inserted into the anatomy. The system or component include one or more features configured to inhibit or prevent unwanted removal of the delivery tool mid procedure and/or prevent significant movement from the external direction that is often associated with patient movement or human error.

In one aspect, there is disclosed an anchor system for securing a delivery tool to skin, comprising: an outer housing forming an internal lumen along an axis, the outer housing having a distal foot with a surface configured to be secured to a patient’s skin; a carriage sleeve slidably positioned within the internal lumen, wherein the carriages sleeve is configured to be attached to the delivery tool; a window on the outer housing, wherein a distance gauge on the carriage sleeve is visible through the window for providing a user an indication of a distance of movement of the carriage sleeve; and an actuator coupled to the outer housing, wherein the actuator is actuated to slidably move the carriage sleeve through the internal lumen.

In another aspect, there is disclosed a method of securing a delivery tool to a patient, comprising: attaching an external anchor system to a skin surface of a patient; inserting a delivery tool into a carriage sleeve of the external anchor system and securing the delivery tool to the carriage sleeve; sliding the delivery tool along an axis defined by the external anchor so that the delivery tool penetrates the skin surface of the patient; and actuating an actuator of the external anchor so that the carriage sleeve and the delivery tool collectively move along the axis.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIGS. 1 and 2 shows perspective views of an example external anchor for a delivery device.

FIG. 3 shows a side view of the external anchor.

FIG. 4 shows an example adhesive base mount for an external anchor.

FIG. 5 shows a Cerebral Spinal Fluid (CSF) catheter system that incorporates an external anchor with micrometer like insertion control and measurement capabilities.

### DETAILED DESCRIPTION

Before the present subject matter is further described, it is to be understood that this subject matter described herein is not limited to particular embodiments described, as such may of course vary. It is also to be understood that the terminology used herein is for the purpose of describing particular embodiments only, and is not intended to be limiting. Unless defined otherwise, all technical terms used herein have the same meaning as commonly understood by one skilled in the art to which this subject matter belongs.

An aspect of the system is configured to enable safe and secured external anchoring of a delivery tool (such as drug delivery tool) inserted into the anatomy. The external anchoring feature stop, inhibits or prevents unwanted removal of the delivery tool mid procedure and prevents significant movement of the tool from the external direction that is often associated with patient movement or human error.

The external anchor can adhere to the patient, such as to the epidermis, through the use of skin safe adhesives. A contacting surface of the external anchor to the epidermis can vary and can be, for example, completely flat. Or the contacting surface of the external anchor can have a contour specifically shaped to the geometry of the epidermis or to a contact surface, such as a table, positioned at the region of interest or the entry point of the tool. The external anchor can be fully fixed onto the delivery tool or a removable accessory that the delivery tool itself is loaded on. Additional to providing external anchoring of the delivery tool in position, the external anchor can also have integrated feature to control delivery tool insertion in a precise manner with visual markers to provide feedback to the user.

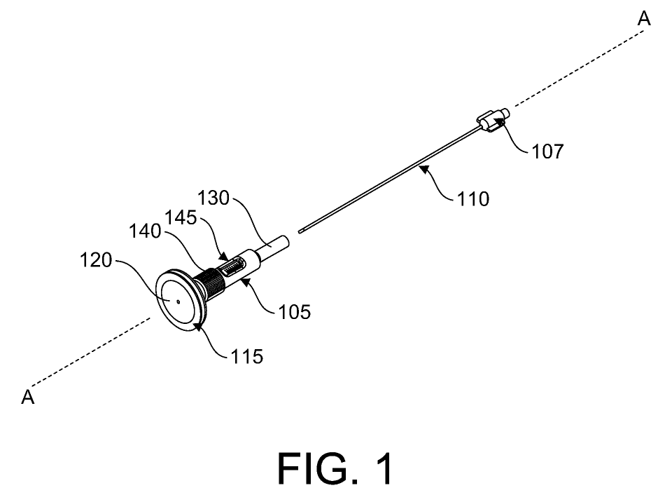

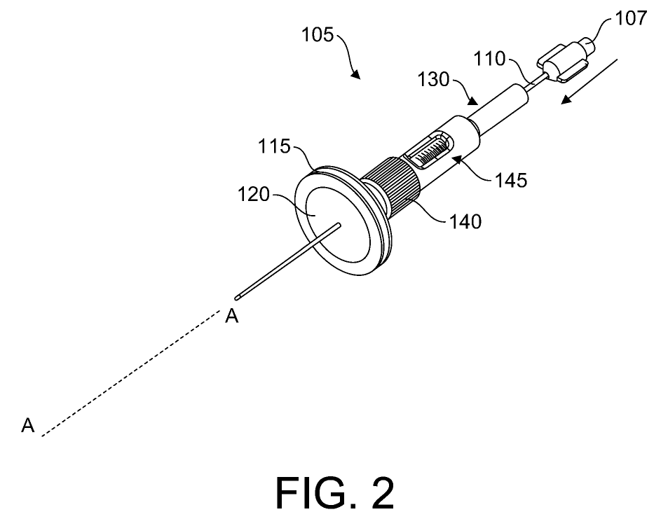

FIGS. 1 and 2 shows perspective views of an example external anchor 105 formed of an outer housing. In FIG. 1, the external anchor 105 is positioned adjacent a delivery device such as a catheter or a spinal needle 110 formed of an elongated body with a sharpened distal tip wherein the needle 110 has a proximal hub or handle 107. FIG. 2 shows the needle 110 mechanically inserted into or coupled to the external anchor 105. The external anchor 105 generally extends along a long axis A-A and has an enlarged distal foot 115 that is flared or has a larger diameter than a proximal region of the external anchor 105. The distal foot 115 has a bottom surface 120 that is configured to be juxtaposed with an outer skin surface of the patient. The distal foot 115 is shown having a flat bottom surface 120 and a circular shape although the contour of the bottom surface and the shape of the distal foot 115 can vary.

The external anchor 105 has an internal passageway that extends along the axis A-A. An elongated carriage sleeve 130 is slidably and movably positioned within the internal passageway. The elongated carriage sleeve 130 has an internal lumen that also extends along the axis A-A. The internal lumen of the carriage sleeve 130 is sized and shaped to receive a delivery tool or delivery device such as the spinal needle 110. It should be appreciated that the delivery device can vary and can be, for example, a catheter such as catheter or other drug delivery device.

The delivery device, once inserted coaxially into the enclosed lumen of the carriage sleeve 130 (as shown in FIG. 2), is secured to the carriage sleeve 130 using mechanical means such as Luer lock(s) between a distal hub of the delivery device and the proximal end of the enclosed lumen of the carriage sleeve 130. A hemostasis valve or rotational vise around the shaft of the delivery tool itself can also be used. The Luer locks secures the delivery tool in a similar fashion as a needle tip to the Luer lock of a syringe, while the hemostasis valve/rotational vise locking mechanisms locks the needle in the same fashion as self-centering rotating chuck but with less destructive pressure at contact points (silicone vs. metal). Both locking mechanisms simultaneously provide a structurally safe way to fixate the position of the delivery device relative to the carriage sleeve 130 and centers relative to the carriage sleeve 130.

The dimensions of the carriage sleeve 130 follows a acceptable ISO standards to retain the flexibility to use a variety of needle devices through this carriage sleeve 130.

With reference still to FIGS. 1 and 2, the external anchor 105 includes an actuator, such as a collar or thimble 140, that is co-axially positioned on the external anchor 105. The thimble 140 is mechanically linked to the carriage sleeve 130 (such as via a threaded and/or a geared coupling) such that actuation (for example, rotation) of the thimble 140 causes linear movement of the carriage sleeve 130. Such linear movement of the carriage sleeve 130 cases corresponding linear movement of the spinal needle 110 (which is secured to the carriage sleeve 130) in a proximal and/or distal direction along the axis A-A. A window 145 extends through the external anchor 105 wherein a gauge or scale is visible through the window 145. A marker on the external anchor 105 can be measured against the scale to provide a user with a visual representation of an amount of linear movement of the carriage sleeve 130. The micrometer like insertion control and measurement capabilities allows the user to use puncture depth as a reference point and for accurate puncture through the dura.

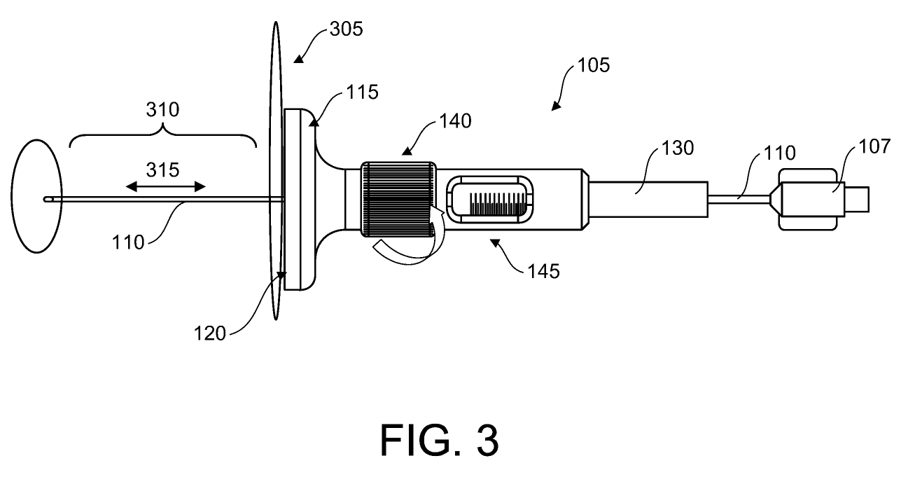

FIG. 3 shows a side view of the external anchor 105 with the foot 115 positioned or otherwise mounted on a skin surface 305 of a patient. The bottom surface 120 of the foot 115 can have an adhesive that secures the foot 115 and the external anchor 105 to the skin. The needle 110 is mounted to the external anchor 105 (such as through the internal lumen of the external anchor 105). A distal region 310 of the needle 110 extends through the skin. The needle 110 is locked to the external anchor 105 specifically to the carriage sleeve 130. The bottom surface 120 can be rigid or it can be flexible or malleable to conform to a skin surface when applied thereto.

As mentioned, a user can actuate the thimble 140 (such as via rotation) to cause corresponding linear movement (as represented by arrow 315) of the needle 110 via the attachment between the needle 110 and the carriage sleeve 130. As mentioned, a gauge or scale is visible through the window 145. The gauge can be viewed in conjunction with a marker on the external anchor 105 to provide a user with an indication of an amount of linear movement of the needle 110 as the thimble 140 is actuated. The provides a measuring a tool for precise insertion control of the needle 110 into the patient.

As the enclosed lumen of the carriage 130 travels coaxially (with the secured delivery tool) to the rest of the injection molded external housing with the larger diameter adhesive covered contact area to the epidermis itself, a cutout window with a marked feature is used in conjunction with the laser etched markings as a measuring a tool for insertion control. In this application, device insertion is precisely controlled through the operation of the thimble that is geared to move the enclosed lumen forward and backward relative to the external housing. The micrometer like insertion control and measurement capabilities allows the user to use puncture depth as a reference point and for accurate puncture through the dura.

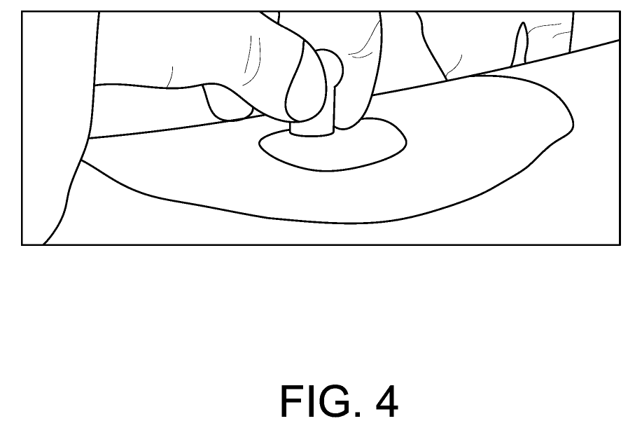

In an alternate embodiment, the external anchor 105 has a plurality of feet (rather than a single foot) each configured to be secured to a patient’s skin or table. This provides additional surface area that adheres to the skin itself or table and spreads the adhesion across a few different components. The system can use one or more flexible skin safe adhesive patches (flexible in its nature to match the geometry of the epidermis from patient to patient) with one or more cut out holes enabling it to be layered over one or more smaller skin safe adhesive rigid base mount components for securing the device to once adhesion to the skin or table has been achieved. FIG. 4 shows an example of this.

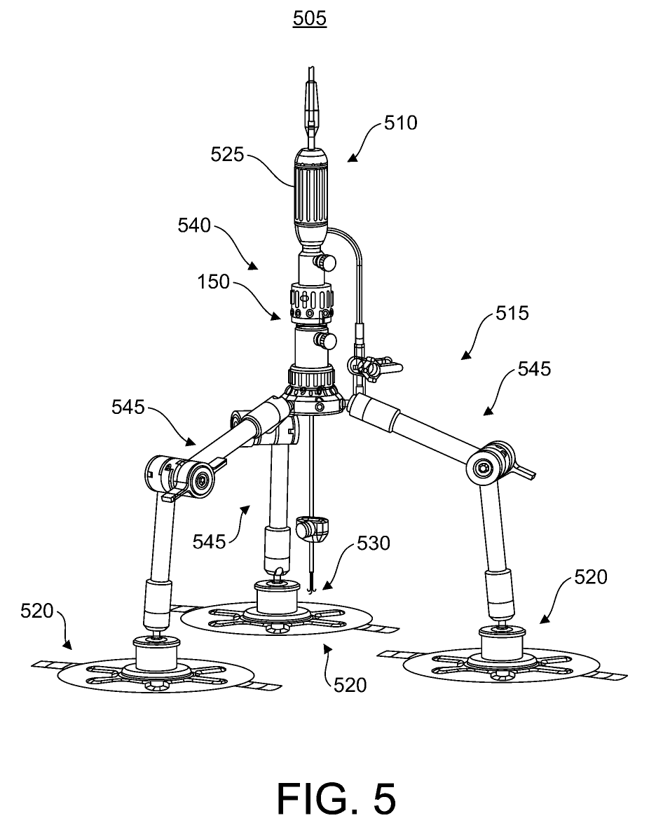

FIG. 5 shows a Cerebral Spinal Fluid (CSF) catheter system 505 that incorporates an external anchor with micrometer like insertion control and measurement capabilities as described above. The system 505 includes a CSF catheter 510 removably coupled to an external support frame 515 that is removably attached to skin of a patient via one or more feet 520 of the support frame 515. The support frame can incorporate the adhesive patches at one or more distal feet 520 of the support frame. As mentioned, an external anchor 150 with micrometer-like linear movement and adjustment capabilities as described above can be incorporated into the support frame 515.

The CSF catheter 510 is an elongated body having at least one internal lumen, such as an injection lumen, wherein fluid can be injected into a region of the patient via the lumen. The CSF catheter 510 has a catheter handle 525 at a proximal region of the catheter. A distal tip of the catheter includes or forms at least one internal anchor 530. In use, the CSF catheter 510 deploys the internal anchor(s) 530 into a CSF space to provide distal support during a procedure. The CSF catheter 510 can also include an aspiration/injection tube configured to aspirate fluid from or inject fluid to the CSF space.

The support frame has a central post region 540 that is attached to and extends upwardly from three or more articulatable legs 545. Each leg 545 is attached to the skin or table at the respective distal foot 520. The external anchor 550 is located at the post region 540 and is sized and shaped to removably receive the CSF catheter 510. As mentioned, the external anchor 550 provides fine placement of the CSF catheter (or other device) relative to patient anatomy and guides the CSF catheter 510. One or more of the legs 545 and/or the distal feet 520 can include or be coupled to an articulation mechanism (such as a ball joint) that can move or articulate about multiple degrees of freedom. The articulation mechanism can also be locked in position. The ball joint can be replaced or supplemented with a system of concentric circular or other tangential geometric magnetic surfaces that freely rotate around each other but remain mechanically intact between its surfaces through magnetism versus mechanical fixation. The ball joints can be coupled to one or more pins that extend towards the ball joints (applying compressive force to create a frictional lock) by a rotational cam at the point where the two support rod portions of the legs meet. A rotational cam creates a compressive force at the ball joint is that a single locking action is used to lock multiple degrees of freedom. The geometry, material, and surface area of both the rigid base mount and flexible adhesive patch components can be further optimized to tune the amount of force the base can absorb before translating force and causing motion of the distal support rod component.

With reference again to FIGS. 1-3, in use the delivery device, such as the needle 110, is inserted into and positioned within the internal passageway of the external anchor 105 by inserting the needle 110 into the elongated carriage sleeve 130. As mentioned, the carriage sleeve 130 is slidably and movably positioned within the internal passageway of the elongated needle 110. This step can occur before or after the external anchor 105 is secured to the skin by positioning the bottom surface 120 on the skin and fixing it thereto with adhesive. Also, the carriage sleeve 130 can be first secured to the needle 110 and the collective structure of the needle 110 and the carriage sleeve 130 can then be coupled to the external anchor 105. Or the needle 110 can be inserted into the carriage sleeve 130 after the carriage sleeve is coupled to the external anchor. As shown in FIGS. 2 and 3, the needle 110 is then advanced along the axis A-A so that it extends through the skin surface 305. The needle 110 can be freely advanced through the external anchor 105 and the position of the needle along the axis A-A can also be adjusted by actuating the thimble 140 such as by rotating the thimble.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. An anchor system for securing a delivery tool to skin, comprising:
an outer housing forming an internal lumen along an axis, the outer housing having a distal foot with a surface configured to be secured to a patient’s skin;
a carriage sleeve slidably positioned within the internal lumen, wherein the carriages sleeve is configured to be attached to the delivery tool;
a window on the outer housing, wherein a distance gauge on the carriage sleeve is visible through the window for providing a user an indication of a distance of movement of the carriage sleeve; and
an actuator coupled to the outer housing, wherein the actuator is actuated to slidably move the carriage sleeve through the internal lumen.

2. The anchor system of claim 1, wherein the surface of the distal foot is flat.

3. The anchor system of claim 1, wherein the surface of the distal foot is contoured.

4. The anchor system of claim 1, wherein the distal foot is a flared body.

5. The anchor system of claim 1, wherein the delivery tool is a spinal needle.

6. The anchor system of claim 1, further comprising an adhesive on the surface of the distal foot, the adhesive configured to adhere the distal foot to skin.

7. The anchor system of claim 1, wherein the actuator is a rotatable thimble.

8. The anchor system of claim 1, wherein the anchor system has a plurality of distal feet each configured to be secured to a patient’s skin.

9. The anchor system of claim 1, wherein each distal foot is coupled to at least one actuating joint configured to adjust a trajectory angle of the delivery device relative to an entry site and a target area.

10. The anchor system of claim 1, wherein the distal foot is detachable.

11. The anchor system of claim 1, wherein the outer housing is configured to lock a position of the delivery tool in place at a target location on the skin or table.

12. A method of securing a delivery tool to a patient, comprising:
attaching an external anchor system to a skin surface of a patient;
inserting a delivery tool into a carriage sleeve of the external anchor system and securing the delivery tool to the carriage sleeve;
sliding the delivery tool along an axis defined by the external anchor so that the delivery tool penetrates the skin surface of the patient; and
actuating an actuator of the external anchor so that the carriage sleeve and the delivery tool collectively move along the axis.

13. The method of claim 12, wherein the delivery tool is inserted into the carriage sleeve while the carriage sleeve is coupled to the external anchor.

14. The method of claim 12, wherein the delivery tool is inserted into the carriage sleeve prior to coupling the carriage sleeve to the external anchor.

15. The method of claim 12, wherein the actuator is a rotatable thimble and wherein actuating the actuator comprises rotating the thimble so that the carriage sleeve and the delivery tool collectively move along the axis.

16. The method of claim 12, wherein the external anchor has a window on an outer housing of the external anchor, wherein a distance gauge on the carriage sleeve is visible through the window for providing a user an indication of a distance of movement of the carriage sleeve.

17. The method of claim 12, wherein the delivery tool is a spinal needle.

18. The method of claim 12, wherein the external anchor system is secured to the skin surface using an adhesive.
